(19) United States
(12) Patent Application Publication
(10) Pub. No.: US 2018/0353394 A1
(43) Pub. Date: Dec. 13, 2018

(54) COSMETIC FOAM MADE FROM AN EMULSION CONTAINING SODIUM CETEARYL SULFATE AND GLYCERYL MONOSTEARATE

(71) Applicant: BEIERSDORF AG, Hamburg (DE)

(72) Inventors: Kerstin SKUBSCH, Prisdorf (DE); Claudia MUELLER, Tangstedt (DE); Sabine SCHULZ, Hamburg (DE); Julia ECKERT, Hamburg (DE); Katrin RUPP, Hamburg (DE)

(21) Appl. No.: 15/772,846

(22) PCT Filed: Oct. 17, 2016
(86) PCT No.: PCT/EP2016/074833

§ 371 Date: May 2, 2018

(30) Foreign Application Priority Data
Nov. 4, 2015 (DE) 10 2015 221 566.0

(51) Int. Cl. A61K 8/04; A61K 8/06; A61K 8/37; A61K 8/46; A61Q 19/00

(52) U.S. Cl. CPC A61K 8/046; A61K 8/062; A61K 8/375; A61K 2800/87; A61Q 19/00; A61K 2800/30; A61K 2800/596; A61K 8/463

(57) ABSTRACT

The invention relates to a cosmetic foam of a) an emulsion containing a combination of sodium cetearyl sulfate and glyceryl monostearate SE and b) a gas or gas mixture of propane, n-butane and/or isobutane, foaming the emulsion, the emulsion being free of polyethylene glycol derivatives (PEG derivatives).

The present invention relates to a cosmetic foam of an emulsion comprising sodium cetearyl sulfate and glyceryl monostearate SE and a gas or gas mixture of propane, n-butane and/or isobutane which foams the emulsion, the emulsion being free of polyethylene glycol derivatives (PEG derivatives). The invention further relates to an aerosol can with release valve containing such a foam.

The desire to look beautiful and attractive is naturally rooted in humans. Although ideals of beauty have changed over time, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Cleansing of the skin serves to remove dirt, sweat and residual dead skin particles, which form an ideal nutrient source for all kinds of germs and parasites. Skincare products mostly serve for moisturizing and refatting the skin. Active ingredients are commonly added thereto, which are intended to regenerate the skin and for example to prevent and reduce the premature aging thereof (e.g. the appearance of fine lines and wrinkles).

Skin care products (also some skin cleansing products) generally consist of emulsions. Emulsions are generally understood to mean heterogeneous systems composed of two liquids that are immiscible, or of only limited miscibility, with each other, which are typically referred to as phases and in which one of the two liquids is dispersed in the other liquid in the form of fine droplets. Externally and viewed with the naked eye, emulsions appear homogeneous.

If the two liquids are water and oil and oil droplets are present finely distributed in water, it is an oil-in-water emulsion (O/W emulsion, milk for example). The basic character of an O/W emulsion is influenced by the water. The reverse principle applies to a water-in-oil emulsion (W/O emulsion, butter for example), wherein the basic character in this case is determined by the oil.

In addition to the “classical” application, where emulsions are applied to the skin directly from the storage container, there are also a lower number of applications in which the emulsion is foamed with the aid of a propellant for application to the skin. Foamed emulsions, by virtue of their foam consistency, have a particular skin feel that is perceived by the users as better distributable on the skin with a lighter and less sticky skin feel. The application of the mousse texture also is fun for the users.

The disadvantage of the prior art, however, is the fact that polyethylene glycols and/or polyethylene glycol derivatives (PEG derivatives; these are compounds having alcohol or acid functions which are fully or partially etherified or esterified) have to be added to the preparations for producing cosmetic foam emulsions, in order to keep the foam stable over a longer period. These PEG derivatives are increasingly undesirable to consumers since some scientists question their harmlessness to health. Whether these concerns have a scientific basis remains an open question in the context of the present invention. The reality, however, is that consumers increasingly demand cosmetics which are “PEG-free”.

It was therefore an object of the invention to develop a sensorially attractive cosmetic foam, based on a foamed emulsion, that is stable over a relatively long period, which is “PEG-free”.

The object is achieved, surprisingly, by a cosmetic foam of

a) an emulsion comprising a combination of sodium cetearyl sulfate and glyceryl monostearate SE and

b) a gas or gas mixture of propane, n-butane and/or isobutane which foams the emulsion, wherein the emulsion is free of polyethylene glycol derivatives (PEG derivatives).

The foam according to the invention is surprisingly uniformly fine-bubbled. It forms what is known as a mousse. The preparation is drawn into the skin surprisingly quickly and leaves behind a pleasant skin feel.

Although EP1277455, EP1014916 and DE10138495 are known to those skilled in the art, these documents could not have indicated the approach of the present invention.

It is of particular advantage in accordance with the invention if the emulsion is free of soaps (an emulsion in accordance with the invention is “free of soaps” if it comprises less than 0.2% by weight of soaps, based on the total weight of the emulsion). In accordance with the invention, “soaps” are understood to mean sodium or potassium salts of fatty acids.

It is likewise of particular advantage in accordance with the invention if the emulsion is free of surfactants having an HLB value of greater than 10, wherein sodium cetearyl sulfate is not counted among the surfactants in accordance with the invention but is a constituent essential to the invention.

This fact is all the more surprising since soaps and surfactants are typically required for stabilizing cosmetic foams. In contrast, omitting the surfactants in the foams according to the invention leads to a stabilization of the foam and to a distinctly creamier foam which is more easily distributed.

In this advantageous embodiment according to the invention, the foams according to the invention are surprisingly more stable and creamier than when these surface-active compounds are added and have a significantly lower potential for skin irritation.

A further advantage according to the invention of this formulation is the long storage stability of the contents (i.e. the emulsion). It is advantageous to use a storage-stable formulation in the production process if the contents cannot be filled immediately into the aerosol bottle. In the case of later bottling, it does not need to be laboriously stirred. In the case of delayed use, the foam of a storage-stable emulsion is easier to shake up in the aerosol can, which is important for a uniform distribution of the propellant gas in the emulsion. Finally, it is ensured that the full fill volume can be readily withdrawn and the propellant gas volume is not prematurely “consumed” by misuse.

It is of advantage in accordance with the invention if the emulsion according to the invention comprises sodium cetearyl sulfate in an amount from 0.1 to 2% by weight, based on the total weight of the emulsion and preferably in accordance with the invention in an amount from 0.1 to 0.5% by weight, based on the total weight of the emulsion.

It is of advantage in accordance with the invention if the emulsion according to the invention comprises glyceryl monostearate SE in an amount of 0.1 to 5% by weight, based on the total weight of the emulsion and preferably in accordance with the invention in an amount from 1 to 3% by weight, based on the total weight of the emulsion.

Self-emulsifying glyceryl monosterate with the INCI Glyceryl Monostearate SE is used in accordance with the invention.

Advantageously in accordance with the invention, the ratio by weight in the emulsion of sodium cetearyl sulfate to glyceryl monostearate is from 1:1 to 1:20.

Embodiments of the present invention that are advantageous according to the invention are characterized in that the foam is formed from 90-96% by weight emulsion and 4 to 10% by weight gas or gas mixture. Preferred embodiments in accordance with the invention are characterized in that the foam is formed from 92-94% by weight emulsion and 6 to 8% by weight gas or gas mixture. That the foams according to the invention can be uniformly foamed even with such low amounts of gas was not foreseeable to those skilled in the art. The reason for this is the surprisingly good and simple uniform distribution of the gas in the emulsion.

It is advantageous in accordance with the invention if the gas used is a gas mixture of butane, isobutane and/or propane. The mixing ratio of the gases is varied depending on the pressure rating, e.g.:

pressure rating 2.7 bar: 60% butane, 20% propane and 20% isobutane.

Pressure rating 3.0 bar: 5.3% butane, 15.3% propane and 79.4% isobutane.

Pressure rating 3.5 bar: 5% butane, 23% propane and 72% isobutane.

Preferred in accordance with the invention are pressure ratings of 2.7 bar, 3.0 bar and 3.5 bar. Particular preference is given to pressure ratings of 3.0 bar and 3.5 bar.

It is particularly advantageous in accordance with the invention if the emulsion comprises ethanol.

In this case, an ethanol content of 1 to 5% by weight, based on the total weight of the emulsion, is preferred according to the invention.

It was particularly surprising in this context that the foam remains stable in the emulsion despite the ethanol content, since it is generally known expertise that ethanol usually destabilizes foams. Moreover, this combination of foam and ethanol has the surprising advantage that the foam is not flammable despite the gas and ethanol content and as a result the customary safety precautions for such products on storage, transport and use are superfluous.

It is of advantage according to the invention if the emulsion is free of parabens, isothiazolinones and 3-iodopropargyl N-butylcarbamate (IPBC).

In contrast, advantageous embodiments according to the invention are characterized in that the emulsion comprises piroctone olamine (1-hydroxy-4-methyl-6-(2,4,4-trimethylpentyl)-2(1H)-pyridone monoethanolamine salt) and/or phenoxyethanol.

If the emulsion comprises piroctone olamine, it is advantageous in accordance with the invention when the piroctone olamine content is from 0.01 to 1% by weight, based on the total weight of the emulsion.

If the emulsion comprises phenoxyethanol, it is advantageous in accordance with the invention when the phenoxyethanol content is from 0.1 to 1% by weight, based on the total weight of the emulsion.

Embodiments of the present invention that are advantageous according to the invention are also obtained when the emulsion comprises EDTA and/or betaine.

If the emulsion contains EDTA, it is advantageous according to the invention when the content of EDTA is from 0.01 to 2% by weight, based on the total weight of the emulsion.

If the emulsion contains betaine, it is advantageous according to the invention when the content of betaine is from 0.001 to 6% by weight, based on the total weight of the emulsion.

Embodiments of the present invention that are advantageous according to the invention are characterized in that the lipid phase of the emulsion comprises one or more fatty alcohols, almond oil, cocoa butter and/or shea butter. It has been particularly surprising in this case that the emulsions, when they comprise stearyl alcohol or behenyl alcohol, result in preparations that are readily and uniformly miscible with the gas, since both fatty alcohols lead to an increase in the viscosity of the emulsion which in principle should impede the miscibility with the gas (gas mixture).

The concentration used advantageously in accordance with the invention of stearyl alcohol is from 0.01 to 4% by weight, based on the total weight of the emulsion.

The concentration used advantageously in accordance with the invention of behenyl alcohol is from 0.01 to 4% by weight, based on the total weight of the emulsion.

The concentration used advantageously in accordance with the invention of almond oil is from 0.01 to 5% by weight, based on the total weight of the emulsion.

The concentration used advantageously in accordance with the invention of cocoa butter is from 0.01 to 5% by weight, based on the total weight of the emulsion.

The concentration used advantageously in accordance with the invention of shea butter is from 0.01 to 5% by weight, based on the total weight of the emulsion.

Embodiments of the present invention that are advantageous according to the invention are characterized in that the proportion by weight of the lipid phase of the emulsion is 7 to 25% by weight, based on the total weight of the emulsion.

It is of advantage in accordance with the invention if the emulsion comprises neither mineral oil nor silicone oil.

Silicone oils may be present however also in amounts of less than 1% by weight, based on the total weight of the emulsion.

It is advantageous in the context of the present invention if the cosmetic foam is characterized in that the emulsion is free of thickeners such as silicon dioxide, aluminum silicates, polysaccharides or derivatives thereof, e.g. hyaluronic acid, xanthan gum, hydroxypropyl methylcellulose, polyacrylates such as carbopols, for example carbomers and acrylates/C10-30 alkyl acrylate crosspolymer.

The water phase of the emulsion according to the invention may comprise customary auxiliaries such as glycerol, electrolytes, self-tanning agents, etc.

It is preferable in accordance with the invention if the cosmetic foam is characterized in that the emulsion comprises from 5 to 15% by weight glycerol, based on the total weight of the emulsion.

The emulsion according to the invention can also advantageously comprise salts, especially sea salt.

It is advantageous in accordance with the invention if the emulsion according to the invention comprises one or more active ingredients selected from the group of the compounds gylcyrrhetic acid, urea, arctiin, alpha-lipoic acid, folic acid, phytoene, D-biotin, coenzyme Q10, hyaluronic acid, alpha-glucosylrutin, carnitine, carnosine, caffeine, natural and/or synthetic isoflavonoids, glycerylglucose, creatine, creatinine, taurine, β-alanine and/or licochalcone A, panthenol, tocopherol, tocopherol acetate, vitamin C, vitamin C derivatives, Glycyrrhiza inflata root extract, magnolia extract.

Moreover, embodiments of the present invention that are advantageous according to the invention are characterized in that the preparation contains propylene glycol, butylene glycol, 2-methylpropan-1,3-diol, ethylhexylglycerol, 1,2-pentanediol, 1,2-hexanediol, 1,2-octanediol and/or 1,2-decanediol. In this case, the content of 2-methylpropan-1,3-diol, 1,2-pentanediol and/or 1,2-hexanediol is preferred according to the invention.

Embodiments of the present invention that are advantageous according to the invention are characterized in that the emulsion comprises one or more UV filters selected from the group of the compounds 2-phenylbenzimidazole-5-sulfonic acid and/or salts thereof; phenylene-1,4-bis(2-benzimidazyl)-3,3′-5,5′-tetrasulfonic acid salts; 1,4-di(2-oxo-10-sulfo-3-bornylidene-methyl)benzene and salts thereof; 4-(2-oxo-3-bornylidenemethyl)benzenesulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; 3-(4-methylbenzylidene)-camphor; 3-benzylidenecamphor; ethylhexyl salicylate; terephthalidenedicamphorsulfonic acid; 2-ethylhexyl 2-cyano-3,3-diphenylacrylate; 2-ethylhexyl 4-(dimethylamino)benzoate; amyl 4-(dimethylamino)benzoate; di(2-ethylhexyl) 4-methoxybenzalmalonate; 2-ethylhexyl 4-methoxycinnamate; isoamyl 4-methoxycinnamate; 2-hydroxy-4-methoxybenzophenone, 2-hydroxy-4-methoxy-4′-methylbenzophenone; 2,2′-dihydroxy-4-methoxybenzophenone; 4-(tert-butyl)-4′-methoxydibenzoylmethane; homomenthyl salicylate; 2-ethylhexyl 2-hydroxybenzoate; dimethicodiethylbenzalmalonate; 3-(4-(2,2-bis ethoxycarbonylvinyl)phenoxy)propenyl)-methoxysiloxane/dimethylsiloxane-copolymer; 4-(tert-butyl)-4′-methoxydibenzoylmethane; hexyl 2-(4′-diethylamino-2′-hydroxybenzoyl)benzoate; dioctylbutylamidotriazone (INCI: Diethylhexyl Butamidotriazone); 2,4-bis[5-1(dimethylpropyl)benzoxazol-2-yl-(4-phenyl)imino]-6-(2-ethylhexyl)imino-1,3,5-triazine with (CAS No. 288254-16-0); 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine); tris(2-ethylhexyl) 4,4′,4″-(1,3,5-triazine-2,4,6-triyltriimino)trisbenzoate (also: 2,4,6-tris[anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; titanium dioxide; zinc oxide.

It is particularly preferable in accordance with the invention if the emulsion is free of propylparaben and butylparaben, 3-iodo-2-propynyl butylcarbamate, 3-(4-methylbenzylidene)camphor, 2-hydroxy-4-methoxybenzophenone (oxybenzone) and halogen-containing compounds.

The cosmetic emulsions according to the invention may also advantageously comprise fillers, although not mandatory, which further improve, for example, the sensory and cosmetic properties of the formulations and which generate or enhance, for example, a velvety or silky skin feel. Advantageous fillers in the context of the present invention are starch and starch derivatives (such as tapioca starch, distarch phosphate, aluminum or sodium starch octenyl succinate and the like), pigments, which have principally neither UV filter nor staining effects, and/or Aerosils® (CAS No. 7631-86-9) and/or talc, polymethylsilsesquioxane, nylon, silica dimethyl silylate.

It is preferable in accordance with the invention if the cosmetic foam is characterized in that the emulsion comprises tapioca starch modified with polymethylsilsequioxane (INCI Tapioca starch+Polymethylsilsesquioxane). This is advantageous in accordance with the invention at a concentration of 0.1 to 3% by weight, based on the total weight of the emulsion.

The foam according to the invention or the emulsion according to the invention advantageously have in accordance with the invention a pH of 5 to 7.5.

Advantageously in accordance with the invention, the foam according to the invention is stored in an aerosol can with release valve and is applied therefrom. For application, the mixture of emulsion and gas(es) according to the invention is firstly mixed thoroughly by shaking in the aerosol can and then withdrawn via the release valve and applied to the skin. In this case, a further advantage in accordance with the invention is shown in that the foam according to the invention can be more completely withdrawn from the aerosol can in comparison with conventional foams, i.e. the so-called “residual emptying” is surprisingly higher than comparable conventional foams. Moreover, the foam according to the invention exhibits a surprisingly good packaging compatibility even at low pH, i.e. corrosion effects, for example, occur less frequently and are weaker than comparable conventional systems.

It is possible to use customary known aerosol can systems as aerosol cans with release valves.

An aerosol can with release valve comprising the foam according to the invention is therefore also in accordance with the invention, and also a method for applying cosmetic foams to the skin, which is characterized in that the mixture of emulsion and gas(es) according to the invention is firstly mixed thoroughly by shaking in the aerosol can and is subsequently withdrawn via the release valve and applied to the skin.

Finally, it is preferable in accordance with the invention if the foam according to the invention is characterized in that it is comprised of an aerosol can with release valve.

Comparative Experiment

| | A | B |
|---|---|---|
| Composition | m [%] | m [%] |
| Isopropyl Palmitate | 3.00 | 3.00 |
| Caprylic/Capric | 3.00 | 3.00 |
| Triglyceride | | |
| Water | 74.40 | 74.40 |
| Sodium Cetearyl Sulfate | 0.15 | |
| Glyceryl Stearate SE | 1.50 | |
| PEG-40 Stearate | | 1.65 |
| Phenoxyethanol | 0.50 | 0.50 |
| Dimethicone | 1.00 | 1.00 |
| Behenyl Alcohol | 2.00 | 2.00 |
| Glycerol | 9.30 | 9.30 |
| 45% aq. Sodium hydroxide | 0.05 | 0.05 |
| Butyrospermum Parkii | 1.00 | 1.00 |
| Butter | | |
| Carbomer | 0.10 | 0.10 |
| C15-19 Alkane | 4.00 | 4.00 |
| Formulation stability | stable | significant water |
| after 2 months' storage | | separation |
| at 40° C. | | (5 mm) |
| Filling in 200 ml can | 94% Emulsion | 94% Emulsion |
| Propellant | 6% | 6% propane/ |
| | propane/butane/ | butane/ |
| | isobutane 3.5 bar* | isobutane 3.5 bar* |
| Appearance of the foam | very creamy, | aqueous, flat, |
| | finely | medium-bubbled, |
| | bubbled, stable | rapidly collapsing |

*= gas pressure rating, see above

### EXAMPLES

The examples below are intended to illustrate the present invention without limiting it. Unless otherwise stated, all quantitative data, fractions and percentages are based on the weight and the total amount or on the total weight of the preparations.

| | C | D | E | F |
|---|---|---|---|---|
| Composition | m [%] | m [%] | m [%] | m [%] |
| Sodium Cetearyl Sulfate | 0.15 | 0.2 | 0.1 | 0.15 |
| Glyceryl Stearate SE | 1.5 | 1 | 2 | 1.5 |
| Isopropyl Stearate | 1 | | | 1 |
| Caprylic/Capric Triglyceride | 3 | 6 | 3 | 3 |
| Octyldodecanol | 4 | | 3 | 4 |
| Isopropyl palmitate | | 3 | 3 | |
| Almond oil | 0.5 | | | |
| Dimethicone | | 1 | | |
| Shea Butter | 1 | 1 | 0.5 | 1 |
| Cocoa butter | | | 0.5 | |
| Tocopherol acetate | | | 0.2 | |
| Sea salt | | 0.1 | | |
| Tapioca starch modified with | 1 | | 1 | |
| polymethylsilsesquioxane | | | | |
| Perfume | 0.3 | 0.35 | 0.3 | 0.35 |
| Glycerol | 5 | 7 | 9 | 10 |
| Phenoxyethanol | 0.8 | 0.6 | 1.0 | 0.75 |
| Piroctone olamine | | 0.05 | | |
| Behenyl alcohol | 2 | 2 | 2 | 2 |
| Ethanol | 1 | 2 | 3 | |
| Sodium EDTA | 0.18 | 0.18 | 0.18 | 0.18 |
| Aqueous sodium hydroxide | pH 5.5 | pH 7.0 | pH 7.0 | pH 6.5 |
| solution/citric acid q.s. | | | | |
| for pH adjustment | | | | |
| Water | to 100 | to 100 | to 100 | to 100 |
| Filling in 200 ml can | 94% | 96% | 95% | 94% |
| | emulsion | emulsion | emulsion | emulsion |
| Propellants | 6% | 4% | 5% | 6% |
| | Propane/ | Propane/ | Propane/ | Propane/ |
| | butane/ | butane/ | butane/ | butane/ |
| | isobutane | isobutane | isobutane | isobutane |
| | 2.7 bar* | 3.0 bar* | 3.0 bar* | 3.5 bar* |

*Gas pressure rating, see above

| | G | H | I | K |
|---|---|---|---|---|
| Composition in % | m [%] | m [%] | m [%] | m [%] |
| Sodium cetearyl sulfate | 0.15 | 0.15 | 0.15 | 0.15 |
| Glyceryl stearate SE | 1.5 | 1 | 2 | 1.5 |
| Mineral oil | 3 | 2 | | 1 |
| Caprylic/Capric Triglyceride | 3 | | 3 | 3 |
| Octyldodecanol | 4 | | 3 | 4 |
| Isopropyl Palmitate | 3 | 3 | 3 | |
| Almond oil | 0.5 | | | |
| Dimethicone | | 1 | | |
| Shea Butter | 1 | | | 1 |
| Vitamin C | 1 | | | |
| Betaine | | 0.1 | 0.5 | |
| Tapioca starch modified with | 1 | | 1 | |
| polymethylsilsesquioxane | | | | |
| Perfume | 0.3 | 0.35 | 0.3 | 0.35 |
| Glycerol | 5 | 7 | 9 | 10 |
| Phenoxyethanol | 0.8 | 0.2 | | 0.75 |
| Stearyl alcohol | 2 | 2 | 2 | 2 |
| Ethanol | 1 | 4 | 3 | 2 |
| Sodium EDTA | 0.18 | 0.18 | 0.18 | 0.18 |
| Aqueous sodium hydroxide | pH 6.5 | pH 7.0 | pH 7.0 | pH 6.5 |
| solution/citric acid for pH | | | | |
| adjustment (quantum satis) | | | | |
| Water | to 100 | to 100 | to 100 | to 100 |
| Filling in 200 ml can | 95% | 94% | 94% | 94% |
| | emulsion | emulsion | emulsion | emulsion |
| Propellants | 5% | 6% | 6% | 6% |
| | Propane/ | Propane/ | Propane/ | Propane/ |
| | butane/ | butane/ | butane/ | butane/ |
| | isobutane | isobutane | isobutane | isobutane |
| | 3.5 bar* | 3.5 bar* | 3.5 bar* | 3.5 bar* |

*Gas pressure rating, see above

1.-17. (canceled)

18. A cosmetic foam, wherein the foam comprises
(i) an emulsion comprising (a) sodium cetearyl sulfate and (b) glyceryl monostearate SE and
(ii) a gas or gas mixture of propane, n-butane and/or isobutane which foams the emulsion;
and wherein (i) is free of polyethylene glycol derivatives (PEG derivatives).

19. The foam of claim 18, wherein (i) is free of soaps and surfactants having an HLB value of greater than 10, sodium cetearyl sulfate not being counted among these surfactants.

20. The foam of claim 18, wherein (a) is present in a concentration of from 0.1% to 2% by weight, based on a total weight of (i).

21. The foam of claim 18, wherein (b) is present in a concentration of from 0.1% to 5% by weight, based on a total weight of (i).

22. The foam of claim 18, wherein a weight ratio (a):(b) is from 1:1 to 1:20.

23. The foam of claim 18, wherein the foam comprises from 90% to 96% by weight of (i) and from 4% to 10% by weight of (ii).

24. The foam of claim 18, wherein the foam comprises from 92% to 94% by weight of (i) and from 6% to 8% by weight of (ii).

25. The foam of claim 18, wherein (i) is free of thickeners.

26. The foam of claim 18, wherein (i) is free of parabens, isothiazolinones and 3-iodopropargyl N-butylcarbamate (IPBC).

27. The foam of claim 18, wherein (i) comprises ethanol.

28. The foam of claim 18, wherein (i) comprises piroctone olamine (1-hydroxy-4-methyl-6-(2,4,4-trimethylpentyl)-2(1H)-pyridone monoethanolamine salt).

29. The foam of claim 18, wherein (i) comprises phenoxyethanol.

30. The foam of claim 18, wherein (i) comprises EDTA and/or betaine.

31. The foam of claim 18, wherein a lipid phase of (i) comprises at least one of stearyl alcohol, behenyl alcohol, almond oil, cocoa butter, shea butter.

32. The foam of claim 18, wherein (i) comprises from 7% to 25% by weight of lipid phase, based on a total weight of (i).

33. The foam of claim 18, wherein (i) is free of mineral oil and silicone oil.

34. The foam of claim 18, wherein (i) comprises tapioca starch modified with polymethylsilsesquioxane (INCI Tapioca starch+Polymethylsilsesquioxane).

35. The foam of claim 18, wherein (i) comprises from 5% to 15% by weight glycerol, based on a total weight of (i).

36. The foam of claim 18, wherein the foam is present in an aerosol can with release valve.

37. An aerosol can with release valve, wherein the can comprises the foam of claim 18.
